(19) United States
(12) Patent Application Publication
(10) Pub. No.: US 2026/0112470 A1
(43) Pub. Date: Apr. 23, 2026

(54) INTELLIGENT ITEM IDENTIFICATION AND EXCHANGE

(71) Applicant: Click to Fill, Inc., Conyers, GA (US)

(72) Inventors: Clint HOUSE, Austin, TX (US); Joseph Edward Rector, Gainesville, GA (US); Jeroen Rinzema, Overijssel (NL); Samuel Whitten, Tampa, FL (US)

(73) Assignee: Click to Fill, Inc., Conyers, GA (US)

(21) Appl. No.: 19/360,375

(22) Filed: Oct. 16, 2025

Related U.S. Application Data
(60) Provisional application No. 63/708,618, filed on Oct. 17, 2024.

(51) Int. Cl. G16H 20/10; G16H 50/30

(52) U.S. Cl. CPC G16H 20/10; G16H 50/30

(57) ABSTRACT

Disclosed herein are system, method, and computer program product embodiments for intelligent item identification and exchange. A computing device may dynamically route prescriptions and related tasks to the most appropriate pharmacy based on real-time data, including inventory levels (e.g., actual and predicted), geographic location, and delivery requirements. The computing device may monitor the prescription processes to identify and generate an alert regarding any adverse drug interactions.

### RELATED APPLICATION

This application claims the benefit of and priority to U.S. Provisional Application No. 63/708,618, filed Oct. 17, 2024, which is incorporated herein by reference in its entirety.

### BACKGROUND

In digital healthcare and telehealth systems the process of managing prescriptions, including their fulfillment and distribution, is often inefficient and disconnected from real-time factors such as medication availability, delivery times, or geographic considerations.

Thus, prescription management and fulfillment are challenging to optimize in real-time while ensuring patient safety. This problem is exacerbated by the need to integrate multiple data points from disparate systems, including pharmacy management systems, delivery services, and drug interaction databases.

### BRIEF DESCRIPTION OF THE DRAWINGS

The accompanying drawings are incorporated herein and form a part of the specification.

FIG. 1 is a block diagram of a system for intelligent item identification and exchange, according to some aspects of this disclosure.

FIG. 2 shows an example system for training a routing module supporting intelligent item identification and exchange, according to some aspects of this disclosure.

FIG. 3 shows a flowchart of an example training method for generating a machine learning classifier used with intelligent item identification and exchange, according to some aspects of this disclosure.

FIG. 4 shows a flowchart of an example method for intelligent item identification and exchange, according to some aspects of this disclosure.

FIG. 5 is an example computer system useful for implementing various aspects of this disclosure.

In the drawings, like reference numbers generally indicate identical or similar elements. Additionally, generally, the leftmost digit(s) of a reference number identifies the drawing in which the reference number first appears.

### DETAILED DESCRIPTION

Provided herein are system, apparatus, device, method, and/or computer program product embodiments, and/or combinations and sub-combinations thereof, for intelligent item identification and exchange. Prescribers, pharmacies, and other related healthcare providers rely on manual or semi-automated systems that do not account for real-time data, such as fluctuating medical inventories or patient-specific delivery needs. As a result, patients can experience delays in receiving their medications, increased costs due to suboptimal routing, and potential risks due to adverse drug interactions. Further, existing electronic medicine and telehealth systems are incapable of handling the complexity of modern prescription management, particularly in the face of increased demand for personalized medication schedules, the proliferation of active pharmaceutical ingredients suppliers, and the increasing complexity of drug interaction databases. These systems also lack the capability to dynamically adjust to real-time changes in drug availability or the patient's circumstances (e.g., moving to a new location). Accordingly, prescription management and fulfillment are challenging to optimize in real-time while ensuring patient safety. This problem is exacerbated by the need to integrate multiple data points from disparate systems, including pharmacy management systems, delivery services, and drug interaction databases.

The system, apparatus, device, method, and/or computer program product embodiments, and/or combinations and sub-combinations thereof, for intelligent item identification and exchange, described herein, address these challenges associated with the technological field of digital healthcare by leveraging advanced algorithms and real-time data to automate the routing of prescriptions to the most suitable pharmacy or supplier. The fulfillment process is continuously monitored, and potential adverse drug interactions are identified, ensuring timely alerts to patients and providers. For example, a prescription management device may include a specially trained module (e.g., a predictive model, machine-learning model, etc.) that may dynamically route prescriptions and related tasks to the most appropriate pharmacy or active pharmaceutical ingredients supplier based on real-time data, including inventory levels, geographic location, and delivery requirements. To ensure that prescriptions may be effectively routed to target destinations, the prescription management device may predict inventory needs across multiple pharmacies based on information including, but not limited to, historical data, current trends, and real-time demand. The system, apparatus, device, method, and/or computer program product embodiments, and/or combinations and sub-combinations thereof, for intelligent item identification and exchange, automate routine tasks, such as prescription verification, inventory checks, and shipment scheduling, reducing manual effort and errors.

The prescription management device may operate as a centralized hub, integrating multiple pharmacy management systems, delivery services, drug interaction databases, and the like to optimize the routing of prescriptions considering factors such as pharmacy/entity functionality and capacity, active pharmaceutical ingredients availability, patient location, and delivery times. The prescription management device may aggregate and analyze data from multiple entities (e.g., pharmacies, active pharmaceutical ingredients suppliers, etc.) and generate real-time analytics regarding operational performance, inventory levels, and patient outcomes. The prescription management device may monitor requests and/or orders for prescription medicine, employ computer vision and semantic analysis to review prescription data/information, and identify potential adverse drug interactions based on prescription data/information and patient history. These and other advantages are described herein.

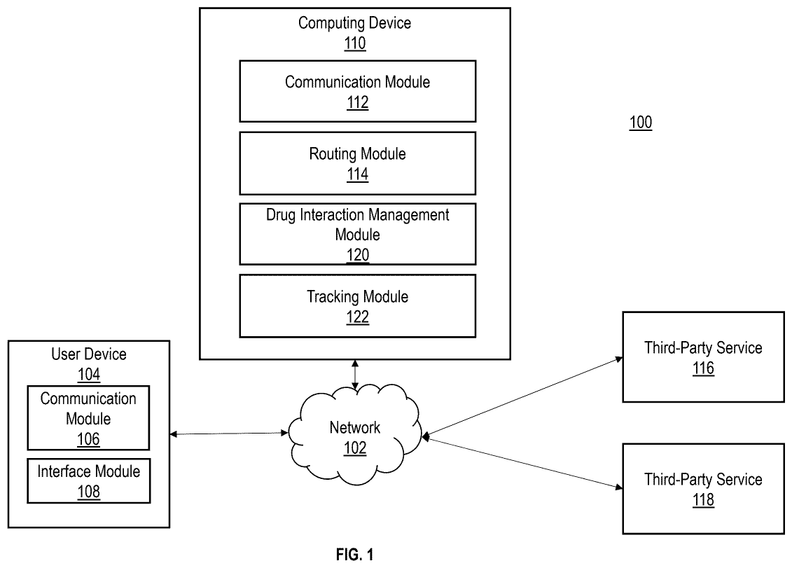

FIG. 1 shows an example system 100 for intelligent item identification and exchange. System 100 is merely an example of one suitable system environment and is not intended to suggest any limitation as to the scope of use or functionality of aspects described herein. System 100 should not be interpreted as having any dependency or requirement related to any single module/component or combination of modules/components described therein.

According to some aspects of this disclosure, system 100 may include a network 102. Network 102 may include a packet-switched network (e.g., internet protocol-based network), a non-packet-switched network (e.g., quadrature amplitude modulation-based network), and/or the like. Network 102 may include network adapters, switches, routers, modems, and the like connected through wireless links (e.g., radiofrequency, satellite) and/or physical links (e.g., fiber optic cable, coaxial cable, Ethernet cable, or a combination thereof). Network 102 may include public networks, private networks, wide area networks (e.g., Internet), local area networks, and/or the like. Network 102 may provide and/or support communication from a telephone, cellular phone, modem, and/or other electronic devices to and throughout the system 100. For example, system 100 may include and support communications between a user device 104, a computing device 110, and third-party services 116 and 118 via network 102.

User device 104 may include a smart device, a mobile device, a computing device, and/or any other device capable of communicating with network 102 and/or device/components in communication with network 102. Although only a single user device 104 is shown, according to some aspects of this disclosure, system 100 may include any number of user devices 104.

User device 104 may include a communication module 106 that facilitates and/or enables communication with network 102 (e.g., devices, components, and/or systems of network 102, etc.), computing device 110, third-party services 116 and 118, and/or any other device/component of the system 100. For example, communication module 106 may include hardware and/or software to facilitate communication. Communication module 106 may comprise one or more of a modem, transceiver (e.g., wireless transceiver, etc.), digital-to-analog converter, analog-to-digital converter, encoder, decoder, modulator, demodulator, tuner (e.g., quadrature amplitude modulation (QAM) tuner, quadrature phase shift keying (QPSK) tuner), and/or the like. Communication module 106 may include any hardware and/or software necessary to facilitate communication.

According to some aspects of this disclosure, user device 104 may include an interface module 108. Interface module 108 enables a user to interact with user device 104, network 102, computing device 110, third-party services 116 and 118, and/or any other device/component of system 100. Interface module 108 may include one or more input/output devices and/or components, for example, such as a display, a keyboard, a pointing device (e.g., a computer mouse, remote control), a microphone, a camera, a joystick, a tactile input device (e.g., touch screen, gloves, etc.), and/or the like. According to some aspects of this disclosure, interaction with the input/output devices and/or components may enable a user to view, access, interact, request, and/or navigate a user interface generated, accessible, and/or displayed by interface module 108. According to some aspects of this disclosure, interaction with the input devices and/or components may enable a user to manipulate and/or interact with components of a user interface, for example, an interactive user interface that supports intelligent item identification and exchange and/or the like.

A user interface that supports intelligent item identification and exchange and/or the like may include a web browser, a website, an application, and the like. The user interface may be used to exchange information between user device 104 and a local source and/or a remote source, such as computing device 110, third-party services 116 and 118, and/or any other device/component of system 100.

According to some aspects of this disclosure, computing device 110 may include a server, a cloud-based compute resource, an entity-controlled device, or any other device capable of communicating with user device 104, third-party services 116 and 118, and/or any other device/component of system 100, either described or (un)shown. Although shown as a single device, according to some aspects of this disclosure, computing device 110 may be part of a computing system and/or infrastructure, and/or may represent a plurality of computing devices. For example, computing device 110 may represent a plurality of computing devices in communication with user device 104, third-party services 116 and 118, and/or any other device/component of system 100.

According to some aspects of this disclosure, computing device 110 may include communication module 112 that facilitates and/or enables communication with network 102 (e.g., devices, components, and/or systems of network 102, etc.), user device 104, third-party services 116 and 118, and/or any other device/component of system 100. For example, communication module 112 may include hardware and/or software to facilitate communication. According to some aspects of this disclosure, communication module 112 may include one or more of a modem, transceiver (e.g., wireless transceiver, etc.), digital-to-analog converter, analog-to-digital converter, encoder, decoder, modulator, demodulator, tuner (e.g., QAM tuner, QPSK tuner), and/or the like. According to some aspects of this disclosure, communication module 112 may include any hardware and/or software necessary to facilitate communication.

According to some aspects of this disclosure computing device 110 may be supported by a cloud-based infrastructure that uses a microservices architecture (e.g., as-a service architecture, etc.) to enable one or more pharmacy management systems, delivery services, and drug interaction databases to communicate on a unified platform. Computing device 110 may operate as an application programming interface (API) gateway and serve as a centralized point for handling requests related to prescribed medicines/drugs, enforcing security policies, orchestrating communication between multiple backend services (such as pharmacies, prescription management systems, and drug databases), and enabling data communication and data exchange between healthcare providers, users/patients, pharmacies, and/or the like. Healthcare providers and prescribers may submit a prescription via an integrated electronic health record (EHR) system and/or the like, computing device 110 may receive the prescription request and route it to a prescription management system (PMS) and inventory API to find the appropriate pharmacy that can fulfill the order based on factors including, but not limited to, inventory, location, and/or the like. Computing device 110 may serve as a load balancer and distribute prescription drug-related requests across multiple backend services (e.g., pharmacies, inventory systems, fulfillment services, etc.).

Computing device 110 may utilize security protocols including, but not limited to, OAuth 2.0, OpenID Connect, and/or the like for user authentication and authorization so that sensitive user/patient data is protected in compliance with standards including, but not limited to, Health Insurance Portability and Accountability Act (HIPAA). Any prescription and/or user-related data/information may be encrypted before being sent to an entity (e.g., pharmacy, etc.) API so that compliance with HIPAA regulations may be maintained.

Computing device 110 may operate to streamline and optimize an item distribution process, such as a prescription fulfillment process, by dynamically routing prescriptions based on a range of real-time factors, including but not limited to, actual and predicted pharmacy inventory levels, geographic considerations, delivery requirements, and pharmacy performance metrics.

Computing device 110 may operate to improve the technological field of digital healthcare and existing prescription management systems by integrating real-time data from pharmacies, active pharmaceutical ingredients suppliers, item/prescription delivery systems, and drug interaction databases. Computing device 110 may facilitate prescription routing, patient data management, real-time communication with physicians and pharmacies, and/or the like. Computing device 110 may operate to ensure that users/patients receive medications in an efficient manner while also safeguarding against adverse reactions. For example, computing device 110 may monitor prescription exchanges and identify potential adverse drug interactions based on information including, but not limited to, prescription data, patient history, and/or the like. Computing device 110 may continuously cross-reference new prescriptions with drug interaction databases and user/patient medical histories and generate real-time alerts if a potential conflict is detected. The status of each prescription from routing through delivery, providing real-time updates to both patients and healthcare providers.

An example operating scenario for computing device 110 is as follows: example user John is prescribed an increased dosage of an example drug. A prescriber my access a user interface an upload the prescription to computing device 110. Computing device 110 may access a user account/profile associated with John and may identify that John has moved to a different state since his last refill. Although there may be a systemic preference value to keeping the same pharmacy that has serviced John in the past for patient expectation and consistency, the pharmacy does not operate in John's new state. Computing device 110 may identify pharmacies that can legally fulfill the prescription in John's new location. Computing device 110 may include a predictive model trained to identify an alternate pharmacy based on a combination of factors including, but not limited to, state certifications, specific formulary match, price, fulfillment/delivery service level agreements (SLAs) and performance metrics, and/or the like. Computing device 110 may identify a pharmacy in proximity to John that has the prescribed drug available for pickup (or delivery) at a cheaper rate. Computing device 110 may check for any potential adverse interactions with John's existing medications. If no conflicts are identified, computing device 110 may route the prescription to the selected pharmacy and send John's user device (e.g., a mobile device, computer, etc.) a notification that the prescribed drug is available for pick up (or delivery). Computing device 110 may forward the prescription to the identified pharmacy and John may be provided instructions on how to obtain the prescribed drug. If selected, John may have the prescribed drug delivered to his location. Computing device 110 may generate real-time notifications about the status of the prescribed drug. For example, John's user device (e.g., a mobile device, computer, etc.) may receive a notification when the prescribed drug is out for delivery and provide information including, but not limited to, an estimated delivery time.

According to some aspects of this disclosure, to facilitate intelligent item exchange, for example, the exchange of medical prescription medicine and/or the like, computing device 110 may include a routing module 114. Routing module 114 may dynamically route items, such as prescriptions and or prescription medicine, to various entities, including, but not limited to, pharmacies, active pharmaceutical ingredients suppliers, and/or the like.

Entities may integrate existing item management systems with routing module 114 via secure application programming interfaces (APIs). For example, third-party services 116 and 118 may represent pharmacies and/or medicine distribution entities. Third-party services 116 and 118 may integrate their pharmacy management system (PMS) with routing module 114 using RESTful APIs, WebSocket APIs, and/or the like. Healthcare providers and users associated with third-party services 116 and 118 may interact with routing module 114 via a web-based portal, mobile application, and/or the like, and may send and receive messages and data through encrypted channels using cryptographic protocols including, but not limited to secure sockets layer (SSL), transport layer security (TLS), and/or the like.

Third-party services 116 and 118 may send/provide routing module 114 information describing which drugs and formularies are in stock and/or provide information detailing prescription medicine inventory levels. According to some aspects of this disclosure, third-party services 116 and 118 may use Internet-of-Things (IoT)-enabled inventory sensors (e.g., cameras, LiDAR devices, computer vision-enabled devices, etc.) that automatically capture sensor/image data indicative of inventory and update stock levels in real time. The IoT devices may be in communication with routing module 114 via a prescription management application and integrated using a messaging protocol (e.g., message queuing telemetry transport, etc.). According to some aspects of this disclosure, third-party services 116 and 118 may send/provide routing module 114 information in a structured data format and/or ark-up language including, but not limited to, Health Level Seven (HL7), JavaScript Object Notation (JSON), Extensible Markup Language (XML), Protobuf, YAML, and/or the like to enable real-time decision-making.

According to some aspects of this disclosure, routing module 114 may include a predictive model (e.g., based on long short-term memory (LSTM) neural networks, etc.) trained to predict/forecast prescription medicine inventory levels, for example, based on information including, but not limited to, historical prescription management data (e.g., historical usage patterns, historical data collected and maintained in a time-series database, etc.), current consumer trends, seasonality, and/or the like. For example, when forecasting inventory levels for a particular pharmacy, routing module 114 may consider that the pharmacy might use more flu medication during winter and, accordingly, may forecast that an inventory for flu medication may be low. Routing module 114 may consider any factors when forecasting inventory levels.

In addition to inventory levels, routing module 114 may consider factors including, but not limited to, geographic location, delivery requirements, and entity performance metrics when facilitating dynamic prescription routing. For example, routing module 114 may consider the geographic location of a user/patient and the location of potential pharmacies (or active pharmaceutical ingredients suppliers) and may prioritize nearby pharmacies when the speed of prescription delivery and/or availability is a factor. Routing module 114 may evaluate prescription medicine delivery speed, cost, and user/patient preferences in real-time. For example, routing analysis may evaluate whether prescription medicines are to be delivered to a user/patient location or retrieved from a pharmacy location.

When routing prescriptions, routing module 114 may utilize an algorithm that evaluates the shortest path from a user/patient (e.g., user device 104, etc.) to the nearest pharmacy (e.g., third-party services 116 and 118, etc.) that can meet inventory and delivery requirements. It calculates not just physical distance but factors in delivery time and pharmacy performance metrics, which are represented as edge weights in the graph.

In an example scenario, if a user/patient of user device 104 needs a specific medication that is in low supply, routing module 114 may check which pharmacies (e.g., third-party services 116 and 118, etc.) have the medication in stock. In a situation where the medication is available via multiple entities, routing module 114 may prioritize an entity (e.g., third-party device 116) that is nearest to the user/patient of user device 104, and select the pharmacy with the best performance metrics for delivery or pickup. If the user/patient of user device 104 has a preferred pharmacy that cannot fulfill the prescription, routing module 114 may identify alternatives and use a large language model (LLM) and/or the like to provide detailed reasoning regarding how alternatives are selected.

According to some aspects of this disclosure, computing device 110 may include a drug interaction management module 120. Drug interaction management module 120 may module monitor new prescriptions for potential drug interactions. For example, drug interaction management module 120 may identify potential adverse drug interactions by cross-referencing data from external drug interaction databases (not shown) in communication with computing device 110. Drug interaction management module 120 may evaluate any user/patient medical history information to identify any known conflicts including, but not limited to, allergies, contraindications, duplication of therapy, formulary adherence, drug interaction, user/patient drug utilization history, FDA-approved uses, side effects, drug to drug interactions and warnings, and/or the like.

For example, if a user/patient is prescribed two medications that are known to interact adversely, drug interaction management module 120 may generate alerts that are sent to the prescribing entity/device, the pharmacy (e.g., third-party service 116 and 118, etc.), and the user/patient (e.g., user device 104, etc.) via real-time notifications. The alert may include options for the prescribing entity/device to adjust the prescription before it is filled and, thus, prevent a harmful interaction.

According to some aspects of this disclosure, computing device 110 may include an item tracking module 122. Item tracking module 122 may monitor a prescription fulfillment process from when routing to the final delivery. Item tracking module 122 may use an API to integrate with the internal systems of third-party services 116 and 118 (e.g., pharmacies, etc.) and external delivery services (e.g., not shown) to provide real-time updates on a user/patient prescription status. GPS-enabled tracking systems configured with prescription couriers may send updates on a prescription delivery status to item tracking module 122 via RESTful APIs. The item tracking module 122 may send prescription delivery status information to a user/patient device (e.g., user device 104, etc.) via push notifications, emails, in-application alerts, and/or the like.

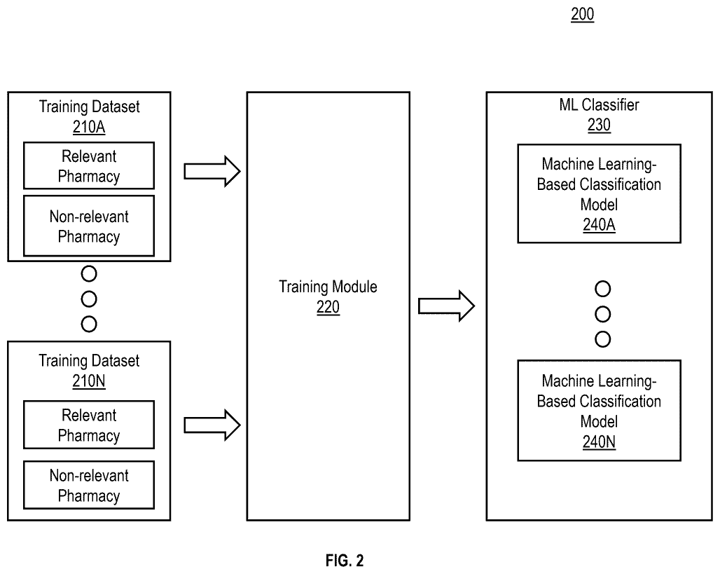

FIG. 2 is described with reference to FIG. 1. As described herein, computing device 110 (e.g., routing module 114) may include a predictive model (e.g., a machine-learning model, etc.) that forecast future shortages routes prescription drug information to enable a user/patient prescription to be filled (e.g., a drug obtained, etc.) based on a combination of factors including, but not limited to, location, state certifications, specific formulary match, inventory, price, fulfillment/delivery service level agreements (SLAs) and performance metrics, and/or the like. According to some aspects of this disclosure, machine learning techniques may be used to train at least one machine learning-based classifier 230 (e.g., a software model, neural network classification layer, etc.) of computing device 110 (e.g., routing module 114). A machine learning-based classifier 230 may be configured by routing module 114 based on an analysis of one or more training datasets 210A-210N.

Training datasets 210A-210N may be generated from data including, but not limited to, historical inventory data for pharmacies and related entities, location/geographic information, pharmacy prescription fulfillment information, and drug cost information, drug interaction information, and/or the like. Training datasets 210A-210N may include metrics related to how users/patients engage with different services/entities (e.g., healthcare providers, pharmacies, etc.) including, but not limited to, service selection, feedback scores, and/or the like. Training datasets 210A-210N may include regulatory information, such as details on healthcare and drug regulations and restrictions relevant to different locations, which could impact the availability of different drugs and/or access to various pharmacies and related entities. Training datasets 210A-210N may also incorporate location-based healthcare regulations and availability data to ensure any prediction or output (e.g., a pharmacy/entity recommendation, etc.) of a trained machine learning model complies with legal restrictions and is geographically relevant. Training datasets 210A-210N may be generated based on information describing any factors relevant to prescription drug distribution and/or fulfillment.

Data for logically linking users/patients to pharmacies for optimal prescription drug exchange may be randomly assigned to a training dataset or a testing dataset. According to some aspects of this disclosure, the assignment of data to a training dataset or a testing dataset may not be completely random. In this case, one or more criteria may be used during the assignment, such as ensuring that similar inventory levels, similar geographic locations, similar prescription drug service level agreement and fulfillment pairings, similar pharmacy and user/patient interaction patterns, similar prescription drug procurement patterns, dissimilar inventory levels, dissimilar geographic locations, dissimilar prescription drug service level agreement and fulfillment pairings, dissimilar pharmacy and user/patient interaction patterns, dissimilar prescription drug procurement patterns, and/or the like may be used in each of the training and testing datasets. In general, any suitable method may be used to assign the data to the training or testing datasets.

The routing module 114 may train the machine learning-based classifier 230 by extracting a feature set from the labeled data according to one or more feature selection techniques. According to some aspects of this disclosure, the routing module 114 may further define the feature set obtained from the labeled data by applying one or more feature selection techniques to the labeled data in one or more training datasets 210A-210N. The routing module 114 may extract a feature set from the training datasets 210A-210N in a variety of ways. The routing module 114 may perform feature extraction multiple times, each time using a different feature-extraction technique. In some instances, the feature sets generated using the different techniques may each be used to generate different machine learning-based classification models 240. According to some aspects of this disclosure, the feature set with the highest quality metrics may be selected for use in training. The routing module 114 may use the feature set(s) to build one or more machine learning-based classification models 240A-240N that are configured to determine and/or predict inventory levels, geographic locations serviced by various pharmacies, prescription drug service level agreement and fulfillment pairings, pharmacy and user/patient interaction patterns, prescription drug procurement patterns, and/or the like.

According to some aspects of this disclosure, the training datasets 210A-210N and/or the labeled data may be analyzed to determine any dependencies, associations, and/or correlations between pharmacies, prescription drug service level agreement and fulfillment pairings, pharmacy and user/patient interaction patterns, prescription drug procurement patterns, and/or the like in the training datasets 210A-210N and/or the labeled data. The term “feature,” as used herein, may refer to any characteristic of an item of data that may be used to determine whether the item of data falls within one or more specific categories.

According to some aspects of this disclosure, a feature selection technique may comprise one or more feature selection rules. One or more feature selection rules may comprise determining which features in the labeled data appear over a threshold number of times in the labeled data and identifying those features that satisfy the threshold as candidate features. For example, any features that appear greater than or equal to 2 times the labeled data may be considered candidate features. Any features appearing less than 2 times may be excluded from consideration as a feature. According to some aspects of this disclosure, a single feature selection rule may be applied to select features or multiple feature selection rules may be applied to select features. According to some aspects of this disclosure, the feature selection rules may be applied in a cascading fashion, with the feature selection rules being applied in a specific order and applied to the results of the previous rule. For example, the feature selection rule may be applied to the labeled data to generate information (e.g., recommended pharmacy for a user/patient to obtain a specific prescription drug, etc.) that may be used for user/patient and pharmacy pairings. A final list of candidate features may be analyzed according to additional features.

According to some aspects of this disclosure, the routing module 114 may generate information (e.g., recommended pharmacy for a user/patient to obtain a specific prescription drug, etc.) that may be used for user/patient and pharmacy pairings based on a wrapper method. A wrapper method may be configured to use a subset of features and train the machine learning model using the subset of features. Based on the inferences that are drawn from a previous model, features may be added and/or deleted from the subset. Wrapper methods include, for example, forward feature selection, backward feature elimination, recursive feature elimination, combinations thereof, and the like. According to some aspects of this disclosure, forward feature selection may be used to identify one or more candidate pharmacies, prescription drug delivery channels, drug-to-drug interaction pairings, user/patient and pharmacy pairings, and/or the like. Forward feature selection is an iterative method that begins with no feature in the machine learning model. In each iteration, the feature that best improves the model is added until the addition of a new variable does not improve the performance of the machine learning model. According to some aspects of this disclosure, backward elimination may be used to identify one or more candidate pharmacies, prescription drug delivery channels, drug-to-drug interaction pairings, user/patient and pharmacy pairings, and/or the like. Backward elimination is an iterative method that begins with all features in the machine learning model. In each iteration, the least significant feature is removed until no improvement is observed in the removal of features. According to some aspects of this disclosure, recursive feature elimination may be used to identify one or more candidate pharmacies, prescription drug delivery channels, drug-to-drug interaction pairings, user/patient and pharmacy pairings, and/or the like. Recursive feature elimination is a greedy optimization algorithm that aims to find the best-performing feature subset. Recursive feature elimination repeatedly creates models and keeps aside the best or the worst-performing feature at each iteration. Recursive feature elimination constructs the next model with the features remaining until all the features are exhausted. Recursive feature elimination then ranks the features based on the order of their elimination.

According to some aspects of this disclosure, one or more candidate pharmacies, prescription drug delivery channels, drug-to-drug interaction pairings, user/patient and pharmacy pairings, and/or the like may be determined according to an embedded method.

Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization, which adds a penalty equivalent to an absolute value of the magnitude of coefficients, and ridge regression performs L2 regularization, which adds a penalty equivalent to the square of the magnitude of coefficients.

After routing module 114 generates a feature set(s), routing module 114 may generate a machine learning-based classification model 240 based on the feature set(s). A machine learning-based predictive model may refer to a complex mathematical model for data classification that is generated using machine-learning techniques. For example, this machine learning-based classifier may include a map of support vectors that represent boundary features. By way of example, boundary features may be selected from and/or represent the highest-ranked features in a feature set.

According to some aspects of this disclosure, the routing module 114 may use the feature sets extracted from the training datasets 210A-210N and/or the labeled data to build a machine learning-based classification model 240A-240N to determine and/or predict pharmacies, prescription drug delivery channels, drug-to-drug interaction pairings, user/patient and pharmacy pairings, and/or the like. According to some aspects of this disclosure, the machine learning-based classification models 240A-240N may be combined into a single machine learning-based classification model 340 (240A-240N). Similarly, the machine learning-based classifier 230 may represent a single classifier containing a single or a plurality of machine learning-based classification models 340 and/or multiple classifiers containing a single or a plurality of machine learning-based classification models 340. According to some aspects of this disclosure, the machine learning-based classifier 230 may also include each of the training datasets 210A-210N and/or each feature set extracted from the training datasets 210A-210N and/or extracted from the labeled data. Although shown separately, routing module 114 may include the machine learning-based classifier 230.

The extracted features from the prescription drug management and fulfillment data may be combined in a classification model trained using a machine learning approach such as discriminant analysis; a decision tree; a nearest neighbor (NN) algorithm (e.g., k-NN models, replicator NN models, etc.); a statistical algorithm (e.g., Bayesian networks, etc.); a clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; a principal component analysis (PCA) (e.g., for linear models); a multi-layer perceptron (MLP) ANNs (e.g., for non-linear models); replicating reservoir networks (e.g., for non-linear models, typically for time series); random forest classification; a combination thereof and/or the like. The resulting machine learning-based classifier 230 may comprise a decision rule or a mapping that uses data associated with various prescription management systems to determine and/or predict pharmacies, prescription drug delivery channels, drug-to-drug interaction pairings, user/patient and pharmacy pairings, and/or the like.

Machine learning-based classifier 230 may be used to determine, predict, and/or identify available pharmacies, prescription drug delivery channels, drug-to-drug interaction pairings, user/patient and pharmacy pairings, and/or the like for the test samples in the test dataset. For example, the result for each test sample may include a confidence level that corresponds to a likelihood or a probability that the corresponding test sample accurately determines and/or predicts pharmacies, prescription drug delivery channels, drug-to-drug interaction pairings, user/patient and pharmacy pairings, and/or the like. The confidence level may be a value between zero and one that represents a likelihood that the determined/predicted pharmacies, prescription drug delivery channels, drug-to-drug interaction pairings, user/patient and pharmacy pairings, and/or the like are consistent with computed values. Multiple confidence levels may be provided for each test sample and each candidate (approximated) pharmacy, prescription drug delivery channel, drug-to-drug interaction pairing, user/patient and pharmacy pairing, and/or the like. A top-performing candidate user intent, communication channel, host domain and services pairing, portal interaction pattern, user interface interaction pattern, and/or the like may be determined by comparing the results obtained for each test sample with computed pharmacies, prescription drug delivery channels, drug-to-drug interaction pairings, user/patient and pharmacy pairings, and/or the like for each test sample. In general, the top-performing candidate pharmacy, prescription drug delivery channel, drug-to-drug interaction pairing, user/patient and pharmacy pairing, and/or the like will have results that closely match the computed pharmacies, prescription drug delivery channels, drug-to-drug interaction pairings, user/patient and pharmacy pairings, and/or the like. The top-performing pharmacy, prescription drug delivery channel, drug-to-drug interaction pairing, user/patient and pharmacy pairing, and/or the like may be used to optimize a prescription delivery and fulfillment process.

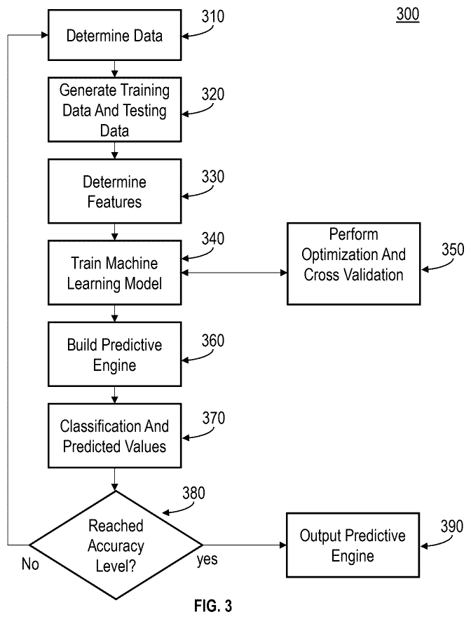

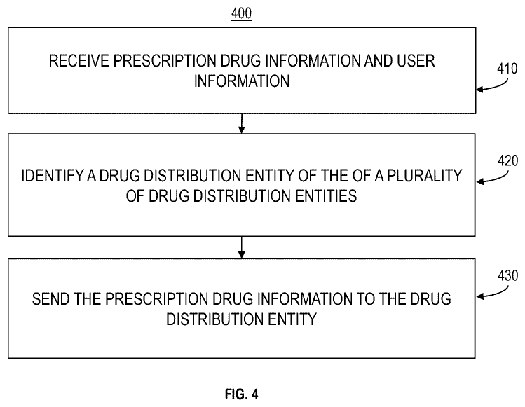

FIG. 3 is a flowchart illustrating an example training method 300. According to some aspects of this disclosure, method 400 configures machine learning classifier 330 for classification through a training process using the routing module 114. The routing module 114 can implement supervised, unsupervised, and/or semi-supervised (e.g., reinforcement-based) machine learning-based classification models 340. Method 300 shown in FIG. 3 is an example of a supervised learning method; variations of this example of training method are discussed below, however, other training methods can be analogously implemented to train unsupervised and/or semi-supervised machine learning (predictive) models. Method 400 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 4, as will be understood by a person of ordinary skill in the art.

Method 300 shall be described with reference to FIGS. 1-3. However, method 300 is not limited to the aspects of those figures.

In 310, the routing module 114 determines, receives, and/or the like, prescription drug management and fulfillment data. Prescription drug management and fulfillment data from various sources may be used to generate one or more datasets, each dataset associated with pharmacies, prescription drug delivery channels, drug-to-drug interaction pairings, user/patient and pharmacy pairings, and/or the like.

In 320, routing module 114 generates a training dataset and a testing dataset.

According to some aspects of this disclosure, the training dataset and the testing dataset may be generated by indicating pharmacies, prescription drug delivery channels, drug-to-drug interaction pairings, user/patient and pharmacy pairings, and/or the like. According to some aspects of this disclosure, the training dataset and the testing dataset may be generated by randomly assigning pharmacies, prescription drug delivery channels, drug-to-drug interaction pairings, user/patient and pharmacy pairings, and/or the like to either the training dataset or the testing dataset. According to some aspects of this disclosure, the assignment of prescription drug management and fulfillment data from various sources as training or test samples may not be completely random. According to some aspects of this disclosure, only the labeled data for a specific feature extracted from a specific prescription drug management and fulfillment data from various sources (e.g., data indicating a preferred pharmacies in a given location, etc.) may be used to generate the training dataset and the testing dataset.

According to some aspects of this disclosure, a majority of the labeled data extracted from prescription drug management and fulfillment data from various sources may be used to generate the training dataset. For example, 75% of the labeled data for determining pharmacies, prescription drug delivery channels, drug-to-drug interaction pairings, user/patient and pharmacy pairings, and/or the like extracted from the prescription drug management and fulfillment data may be used to generate the training dataset and 25% may be used to generate the testing dataset. Any method or technique may be used to create the training and testing datasets.

In 330, routing module 114 determines (e.g., extract, select, etc.) one or more features that can be used by, for example, a classifier (e.g., a software model, a classification layer of a neural network, etc.) to label features extracted from a variety of prescription drug management and fulfillment data and/or data from various sources. One or more features may comprise indications of pharmacies, prescription drug delivery channels, drug-to-drug interaction pairings, user/patient and pharmacy pairings, and/or the like. According to some aspects of this disclosure, the routing module 114 may determine a set of training baseline features from the training dataset. Features of prescription drug management and fulfillment data may be determined by any method.

In 340, routing module 114 trains one or more machine learning models, for example, using one or more features. According to some aspects of this disclosure, the machine learning models may be trained using supervised learning. According to some aspects of this disclosure, other machine learning techniques may be employed, including unsupervised and semi-supervised learning. The machine learning models trained in 340 may be selected based on different criteria and/or data available in the training dataset. For example, machine learning classifiers can suffer from different degrees of bias. According to some aspects of this disclosure, more than one machine learning model can be trained.

In 350, routing module 114 optimizes, improves, and/or cross-validates trained machine-learning models. For example, data for training datasets and/or testing datasets may be updated and/or revised to include more labeled data indicating different pharmacies, prescription drug delivery channels, drug-to-drug interaction pairings, user/patient and pharmacy pairings, and/or the like.

In 360, routing module 114 selects one or more machine-learning models to build a machine-learning model (e.g., a machine-learning classifier, a predictive engine, etc.). The machine-learning model may be evaluated using the testing dataset.

In 370, routing module 114 executes the machine-learning model to analyze the testing dataset and generate classification values and/or predicted values.

In 380, routing module 114 evaluates classification values and/or predicted values output by the machine-learning model to determine whether such values have achieved the desired accuracy level. The performance of the machine-learning model may be evaluated in several ways based on a number of true positive, false positive, true negative, and/or false negative classifications of the plurality of data points indicated by the machine-learning model. For example, the false positives of the machine-learning model may refer to the number of times the machine-learning model incorrectly predicted and/or determined pharmacies, prescription drug delivery channels, drug-to-drug interaction pairings, user/patient and pharmacy pairings, and/or the like. Conversely, the false negatives of the machine-learning model may refer to the number of times the machine-learning model predicted and/or determined pharmacies, prescription drug delivery channels, drug-to-drug interaction pairings, user/patient and pharmacy pairings, and/or the like incorrectly, when in fact, the predicted and/or determined pharmacies, prescription drug delivery channels, drug-to-drug interaction pairings, user/patient and pharmacy pairings, and/or the like matches actual pharmacies, prescription drug delivery channels, drug-to-drug interaction pairings, user/patient and pharmacy pairings, and/or the like. True negatives and true positives may refer to the number of times the machine-learning model correctly predicted and/or determined pharmacies, prescription drug delivery channels, drug-to-drug interaction pairings, user/patient and pharmacy pairings, and/or the like. Related to these measurements are the concepts of recall and precision. Generally, recall refers to a ratio of true positives to a sum of true positives and false negatives, which quantifies the sensitivity of the machine-learning model. Similarly, precision refers to a ratio of true positives as a sum of true and false positives.

In 390, routing module 114 outputs the machine-learning model (and/or an output of the machine-learning model). For example, routing module 114 may output the machine-learning model when such a desired accuracy level is reached. An output of the machine-learning model may end the training phase.

According to some aspects of this disclosure, when the desired accuracy level is not reached, in 390, routing module 114 may perform a subsequent iteration of the training method 300 starting at 310 with variations such as, for example, considering a larger collection of prescription drug management and fulfillment data and/or data from various sources (e.g., third-party services 116 and 118 of FIG. 1.).

FIG. 4 shows a flowchart of an example method 400 for intelligent item identification and exchange, according to some aspects of this disclosure. Method 400 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 4, as will be understood by a person of ordinary skill in the art. Method 400 shall be described with reference to FIGS. 1-3. However, method 400 is not limited to the aspects of those figures.

In 410, computing device 110 receives prescription drug information and user information.

In 420, computing device 110 identifies a drug distribution entity of a plurality of drug distribution entities based on respective drug inventory information and respective prescription drug fulfillment information for each drug distribution entity of the plurality of drug distribution entities and a location indicated by the user information. The respective prescription drug fulfillment information for each drug distribution entity of the plurality of drug distribution entities may include, but is not limited to, an indication of a previous prescription drug delivery time, pricing information for the previous prescription drug, a user satisfaction information for the previous prescription drug, and/or the like.

According to some aspects of this disclosure, computing device 110 may identify the drug distribution entity of the plurality of drug distribution entities by inputting the respective drug inventory information and respective prescription drug fulfillment information for each drug distribution entity of the plurality of drug distribution entities and a location indicated by the user information into a predictive model trained to identity drug distribution entities based on contextual factors. The predictive model may output an indication of the drug distribution entity of the plurality of drug distribution entities.

In 430, computing device 110 sends the prescription drug information to the drug distribution entity.

According to some aspects of this disclosure, the method 400 may further include computing device 110 tracking a fulfillment status for a prescription drug indicated by the prescription drug information.

According to some aspects of this disclosure, the method 400 may further include computing device 110 comparing the prescription drug information to a drug interaction database and a user medical record. Computing device 110 may identify a potential adverse drug interaction. Computing device 110 may send a notification to a user device that indicates that a prescription drug indicated by the prescription drug information is available via the drug distribution entity.

According to some aspects of this disclosure, the method 400 may further include computing device 110 sending a user device associated with the prescription drug information an notification of a potential adverse drug interaction.

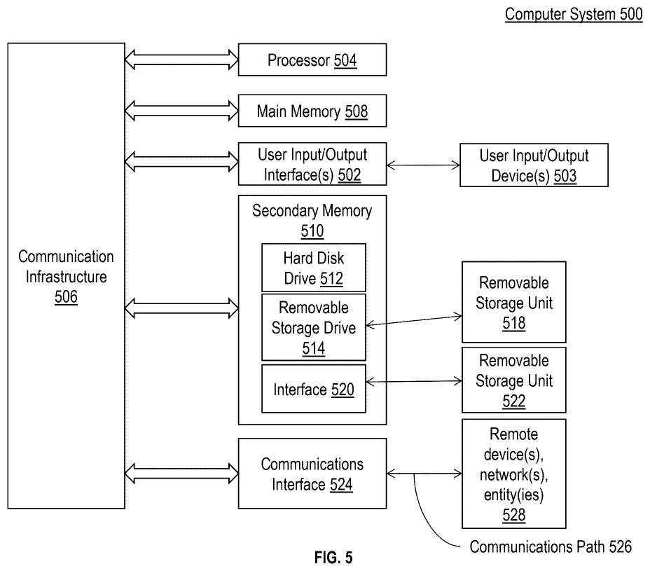

Various embodiments may be implemented, for example, using one or more well-known computer systems, such as computer system 500 shown in FIG. 5. For example, user device 104, computing device 110, third-party services 116 and 118, and/or any other device/component described herein may be implemented using combinations or sub-combinations of computer system 500. Also or alternatively, one or more computer systems 500 may be used, for example, to implement any of the embodiments discussed herein, as well as combinations and sub-combinations thereof.

Computer system 500 may include one or more processors (also called central processing units, or CPUs), such as a processor 504. Processor 504 may be connected to a communication infrastructure or bus 506.

Computer system 500 may also include user input/output device(s) 503, such as monitors, keyboards, pointing devices, etc., which may communicate with communication infrastructure 506 through user input/output interface(s) 502.

One or more of processors 504 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 500 may also include a main or primary memory 508, such as random access memory (RAM). Main memory 508 may include one or more levels of cache. Main memory 508 may have stored therein control logic (i.e., computer software) and/or data.

Computer system 500 may also include one or more secondary storage devices or memory 510. Secondary memory 510 may include, for example, a hard disk drive 512 and/or a removable storage device or drive 514. Removable storage drive 514 may be a floppy disk drive, a magnetic tape drive, a compact disk drive, an optical storage device, a tape backup device, and/or any other storage device/drive.

Removable storage drive 514 may interact with a removable storage unit 518.

Removable storage unit 518 may include a computer-usable or readable storage device having stored thereon computer software (control logic) and/or data. Removable storage unit 518 may be a floppy disk, magnetic tape, compact disk, DVD, optical storage disk, and/or any other computer data storage device. Removable storage drive 514 may read from and/or write to removable storage unit 518.

Secondary memory 510 may include other means, devices, components, instrumentalities, or other approaches for allowing computer programs and/or other instructions and/or data to be accessed by computer system 500. Such means, devices, components, instrumentalities, or other approaches may include, for example, a removable storage unit 522 and an interface 520. Examples of the removable storage unit 522 and the interface 520 may include a program cartridge and cartridge interface (such as that found in video game devices), a removable memory chip (such as an EPROM or PROM) and associated socket, a memory stick and USB or other port, a memory card and associated memory card slot, and/or any other removable storage unit and associated interface.

Computer system 500 may further include a communication or network interface 524. Communication interface 524 may enable computer system 500 to communicate and interact with any combination of external devices, external networks, external entities, etc. (individually and collectively referenced by reference number 528). For example, communication interface 524 may allow computer system 500 to communicate with external or remote devices 528 over communications path 526, which may be wired and/or wireless (or a combination thereof), and which may include any combination of LANs, WANs, the Internet, etc. Control logic and/or data may be transmitted to and from computer system 500 via communication path 526.

Computer system 500 may also be any of a personal digital assistant (PDA), desktop workstation, laptop or notebook computer, netbook, tablet, smartphone, smartwatch or other wearable, appliance, part of the Internet-of-Things, and/or embedded system, to name a few non-limiting examples, or any combination thereof.

Computer system 500 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 500 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats, or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 500, main memory 508, secondary memory 510, and removable storage units 518 and 522, as well as tangible articles of manufacture embodying any combination of the foregoing. Such control logic, when executed by one or more data processing devices (such as computer system 500 or processor(s) 504), may cause such data processing devices to operate as described herein.

Based on the teachings contained in this disclosure, it will be apparent to persons skilled in the relevant art(s) how to make and use embodiments of this disclosure using data processing devices, computer systems, and/or computer architectures other than that shown in FIG. 5. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expressions “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A computer-implemented method for intelligent item identification and exchange, comprising:
receiving prescription drug information and user information;
identifying a drug distribution entity of the of a plurality of drug distribution entities based on respective drug inventory information and respective prescription drug fulfillment information for each drug distribution entity of the plurality of drug distribution entities and a location indicated by the user information; and
sending the prescription drug information to the drug distribution entity.

2. The computer-implemented method of claim 1, further comprising tracking a fulfillment status for a prescription drug indicated by the prescription drug information.

3. The computer-implemented method of claim 1, wherein the respective prescription drug fulfillment information for each drug distribution entity of the plurality of drug distribution entities comprises at least one of an indication of a previous prescription drug delivery time, pricing information for the previous prescription drug, or user satisfaction information for the previous prescription drug.

4. The computer-implemented method of claim 1, further comprising sending a notification to a user device that indicates that a prescription drug indicated by the prescription drug information is available via the drug distribution entity.

5. The computer-implemented method of claim 1, further comprising:
comparing the prescription drug information to a drug interaction database and a user medical record; and
identifying a potential adverse drug interaction.

6. The computer-implemented method of claim 5, further comprising sending a user device associated with the prescription drug information a notification of the potential adverse drug interaction.

7. The computer-implemented method of claim 1, wherein the identifying the drug distribution entity of the plurality of drug distribution entities further comprises:
inputting the respective drug inventory information and respective prescription drug fulfillment information for each drug distribution entity of the plurality of drug distribution entities and a location indicated by the user information into a predictive model trained to identity drug distribution entities based on contextual factors; and
receiving an indication of the drug distribution entity of the plurality of drug distribution entities from the predictive model.

8. A system for intelligent item identification and exchange, comprising:
one or more memories;
at least one processor each coupled to at least one of the memories and configured to perform operations comprising:
receiving prescription drug information and user information;
identifying a drug distribution entity of the of a plurality of drug distribution entities based on respective drug inventory information and respective prescription drug fulfillment information for each drug distribution entity of the plurality of drug distribution entities and a location indicated by the user information; and
sending the prescription drug information to the drug distribution entity.

9. The system of claim 8, the operations further comprising tracking a fulfillment status for a prescription drug indicated by the prescription drug information.

10. The system of claim 8, wherein the respective prescription drug fulfillment information for each drug distribution entity of the plurality of drug distribution entities comprises at least one of an indication of a previous prescription drug delivery time, pricing information for the previous prescription drug, or user satisfaction information for the previous prescription drug.

11. The system of claim 8, the operations further comprising sending a notification to a user device that indicates that a prescription drug indicated by the prescription drug information is available via the drug distribution entity.

12. The system of claim 8, the operations further comprising:
comparing the prescription drug information to a drug interaction database and a user medical record; and
identifying a potential adverse drug interaction.

13. The system of claim 12, the operations further comprising sending a user device associated with the prescription drug information a notification of the potential adverse drug interaction.

14. The system of claim 12, wherein the identifying the drug distribution entity of the plurality of drug distribution entities further comprises:
inputting the respective drug inventory information and respective prescription drug fulfillment information for each drug distribution entity of the plurality of drug distribution entities and a location indicated by the user information into a predictive model trained to identity drug distribution entities based on contextual factors; and
receiving an indication of the drug distribution entity of the plurality of drug distribution entities from the predictive model.

15. A non-transitory computer-readable medium having instructions stored thereon that, when executed by at least one computing device, causes the at least one computing device to perform operations comprising:
receiving prescription drug information and user information;
identifying a drug distribution entity of the of a plurality of drug distribution entities based on respective drug inventory information and respective prescription drug fulfillment information for each drug distribution entity of the plurality of drug distribution entities and a location indicated by the user information; and
sending the prescription drug information to the drug distribution entity.

16. The non-transitory computer-readable medium of claim 15, the operations further comprising tracking a fulfillment status for a prescription drug indicated by the prescription drug information.

17. The non-transitory computer-readable medium of claim 15, wherein the respective prescription drug fulfillment information for each drug distribution entity of the plurality of drug distribution entities comprises at least one of an indication of a previous prescription drug delivery time, pricing information for the previous prescription drug, or user satisfaction information for the previous prescription drug.

18. The non-transitory computer-readable medium of claim 15, the operations further comprising sending a notification to a user device that indicates that a prescription drug indicated by the prescription drug information is available via the drug distribution entity.

19. The non-transitory computer-readable medium of claim 15, the operations further comprising:
comparing the prescription drug information to a drug interaction database and a user medical record; and
identifying a potential adverse drug interaction.

20. The non-transitory computer-readable medium of claim 19, the operations further comprising sending a user device associated with the prescription drug information a notification of the potential adverse drug interaction.
